(19) United States
(12) Patent Application Publication
(10) Pub. No.: US 2015/0018575 A1
(43) Pub. Date: Jan. 15, 2015

(54) HIGHLY SEGREGATED JET MIXER FOR PHOSGENATION OF AMINES

(71) Applicant: Dow Global Technologies LLC, Midland, MI (US)

(72) Inventors: Paul A. Gillis, Lake Jackson, TX (US); Joydeep Mukherjee, Missouri City, TX (US); Paulo Pereira, Lake Jackson, TX (US)

(21) Appl. No.: 14/344,345

(22) PCT Filed: Sep. 20, 2012
(86) PCT No.: PCT/US12/56341

§ 371 Date: Mar. 12, 2014

Related U.S. Application Data
(60) Provisional application No. 61/541,673, filed on Sep. 30, 2011.

(51) Int. Cl. B01F 5/04; C07C 263/10

(52) U.S. Cl. CPC B01F 5/04; C07C 263/10
USPC 560/336; 366/173.1

(57) ABSTRACT

Embodiments of the present invention provide a mixing conduit (100) having at least a cylindrical inner surface or a cylindrical outer surface and increased number of jet openings (102). The mixing conduits according to embodiment of the present invention improve mixing rates thus reducing formation of undesired by-products without sacrificing structural integrity. Particularly, embodiments of the present invention provide a static mixer (150) having a substantially circular mixing conduit (100) with more than about 22 jet openings.

### BACKGROUND OF THE INVENTION

1. Field of the Invention

Embodiments of the present invention relate to a mixing apparatus for mixing fluid components, such as the mixing of phosgene and amine in a reactive chemical process.

2. Description of the Related Art

The field of conventional mixing devices can be roughly divided into two main areas: dynamic or mechanical mixers and static mixers. Dynamic or mechanical mixers rely on some type of moving part or parts to ensure the desired or thorough mixing of the components. Static mixers generally have no prominent moving parts and instead rely on flow profiles and pressure differentials within the fluids being mixed to facilitate mixing. The current disclosure is mostly directed to a static mixer but could also be used in combination with dynamic mixers.

Isocyanates are molecules characterized by N═C═O functional groups. The most widely used isocyanates are aromatic compounds. Two aromatic isocyanates are widely produced commercially, namely, toluene diisocyanate (TDI) and methylene diphenyl diisocyanate (MDI). Isocyanates may be reacted with polyols to form polyurethanes. Major polyurethane applications are rigid foams, which are good insulators and are heavily used in appliance, automotive and construction businesses; and flexible foams which may be used in mattresses and furniture applications. In addition aliphatic isocyanates such as hexamethylene diisocyanates are also widely produced and used in special applications such as abrasion and UV resistant coatings.

Mixing of phosgene and amine is an important step in isocyanate production process. The isocyanate product quality and yield are dependent on the efficiency of the mixing and subsequent reaction process involving two continuous streams of reactants, i.e. amine and phosgene. Secondary reactions like the reaction between phosgenation products and amine to form ureas and other urea derivatives ultimately reduce the quality and yield of the production process. While the production of isocyanates is desired, secondary reactions lead to the creation of undesired products, such as ureas and urea derivatives like carbodiimides, and uretonimines The overall chemical reaction can be depicted as follows:

Amine+Phosgene→Isocyanate+Hydrochloric Acid+Ureas+Carbodiimides+Uretonimines+Undesired products

While many known and unknown factors control the quality of the principal reaction, an increase of the ratio of phosgene to amine, a dilution of amine in a solvent, or efficient mixing, minimizes the formation of undesired by-products. Some of the undesired byproducts may be solids and are associated with fouling of the process equipment utilized in the production process.

Consequently, mixer designs with improper mixing can result in lower overall yield of the desired product or can generate a product that clogs or fouls the reactor system leading to down time and/or increased maintenance costs.

FIG. 1 illustrates a partial sectional perspective view of a cylindrical conduit 3 where a phosgene flow 1 goes from the right to the left and amine flows 2 are injected into the phosgene flow 1 through jet openings 4 drilled through the cylindrical conduit 3. Traditionally, only a few jet openings 4 are created in the conduit 3. However, traditional static mixtures often generate undesired by-products due to inefficient mixing.

Zaby et al. (U.S. Pat. No. 5,117,048) indicates that the number of jet openings is limited by diameters of the conduit and the jet openings, and provides conduits with 6 to 12 jet openings (claim 9 and Examples 1-6). Shang et al. (US Publication 2011/0124907) teaches a cylindrical conduit having 2-20 jet openings (claim 2).

Ding et al. (US Publication 2008/0159065) teaches a rectangular shaped conduit having 22, 24, or 52 jet openings (FIGS. 4-6, examples 1-4). Ding indicates using rectangular shaped conduits with large aspect ratios, wherein one dimension of the cross-section is substantially larger than the other, can house the above mentioned number of jet openings to improve mixing of the jets. However, rectangular shaped conduits are impractical for installation due to high differential pressure between the interior and exterior of the phosgene conduit and substantial structural stresses at the bends necessitating a substantially thick mixing conduit.

It would be desirable to have a static mixer that improves phosgene and amine mixing thus limiting the production of undesired by-products.

### SUMMARY OF THE INVENTION

Embodiments of the present invention relate to a static mixing apparatus that can be used alone or in combination with dynamic mixers.

One embodiment of the static mixing apparatus provides a mixing conduit. The mixing conduit comprises a sidewall surrounding an axis and enclosing an inner volume along the axis. The inner volume has two openings on opposite ends allowing an axial stream to enter and exit the inner volume along the axis. The sidewall has an inner surface facing the inner volume and an outer surface facing an exterior. At least one of a cross section of the inner surface and a cross section of the outer surface is circular. A plurality of jet openings are formed through the sidewall in a plane perpendicular to the axis. The plurality of jet openings allow lateral streams to flow into the inner volume, and the number of the plurality of jet openings is greater than 20.

Another embodiment of the present invention provides a static mixer comprising a mixing conduit according to embodiment of the present invention.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 schematically illustrates flow arrangements in a typical static mixer of prior art used in mixing phosgene and amine.

FIG. 2 is a sectional view of a static mixture according to one embodiment of the present invention.

FIG. 3A is a sectional view of a mixing conduit according to one embodiment of the present invention.

FIG. 3B is a side view of the mixing conduit of FIG. 3A.

FIG. 4 is a graph showing simulated mixer performance of static mixers shown in FIG. 2.

FIG. 5 is a sectional view of a static mixture according to another embodiment of the present invention.

FIG. 6 is a graph showing predicted simulation mixer performance of static mixtures shown in FIG. 5.

FIG. 7 is a sectional view of a mixing conduit according to one embodiment of the present invention.

FIG. 8 is a sectional view of a mixing conduit according to another embodiment of the present invention.

FIG. 9 is a sectional view of a mixing conduit according to another embodiment of the present invention.

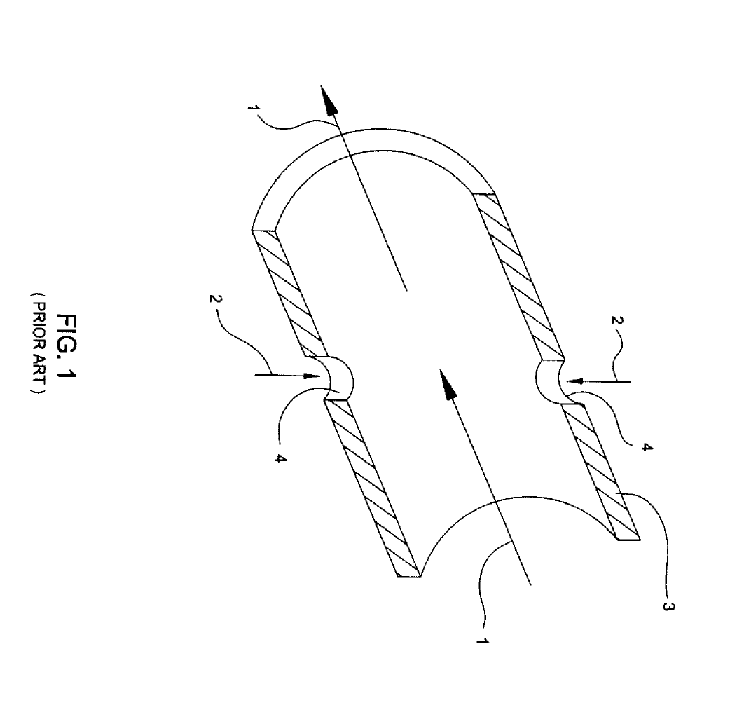

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Embodiments of the present invention relate to a static mixing apparatus for mixing components, in applications with or without chemical reactions, where mixing is rate-limiting step and may cause undesired by-product formation. Embodiments of the present invention provide a mixing apparatus for mixing fluid components such as phosgene and amine during a highly reactive chemical reaction.

Embodiments of the present invention create a velocity profile in a first flow, typically a main cross-flow, as the first flow passes through a mixing conduit and intersects with a second flow injected into the mixing conduit by one or more jet openings formed through the mixing conduit.

Embodiments of the present invention provide a mixing conduit having at least a cylindrical inner surface and/or a cylindrical outer surface and increased number of jet openings. The mixing conduits according to embodiment of the present invention improve mixing rates thus reducing formation of undesired by-products without sacrificing structural integrity. Particularly, embodiments of the present invention provide a static mixer having a substantially circular mixing conduit with more than about 20.

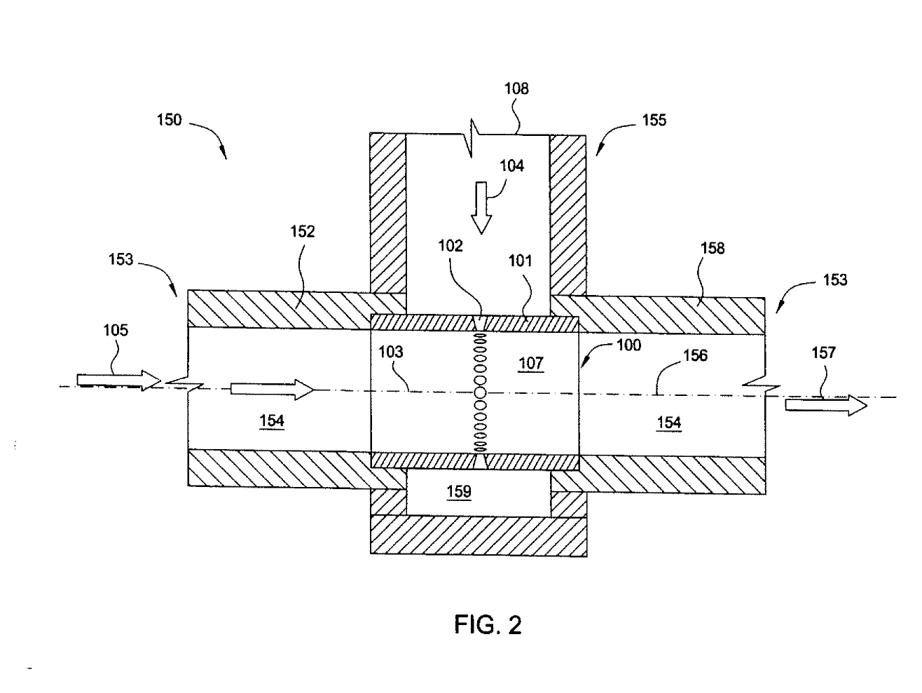

FIG. 2 is a sectional view of a static mixer 150 according to one embodiment of the present. The static mixer 150 comprises a first flow conduit 153 defining an inner volume 154 which allows a first flow 105 therethrough along a longitudinal axis 156. In one embodiment, the first flow conduit 153 comprises an inlet end 152 and an outlet end 158.

A mixing conduit 100 is coupled between the inlet end 152 and the outlet end 158 of the first flow conduit 153. The mixing conduit 100 comprises a sidewall 101 surrounding a central axis 103. The mixing conduit 100 is positioned so that the central axis 103 coincides with the longitude axis 156 of the first flow conduit 153. The sidewall 101 defines an inner volume 107 co-axial with the inner volume 154 of the first-flow conduit 153. A plurality of jet openings 102 are formed through the sidewall 101 fluidly connecting the inner volume 107 of the mixing conduit 100 to an exterior of the mixing conduit 100.

A second flow conduit 155 is attached to the first flow conduit 153. The second flow conduit 155 is coupled to the inlet end 152 and the outlet end 158 of the first flow conduit 153 and defines an annular chamber 159 surrounding the mixing conduit 100. The annular chamber 159 allows a second flow 104 to enter the inner volume 107 of the mixing conduit 100 through the plurality of jet openings 102 and to mix with the first flow 105.

The first flow 105 enters the static mixer 150 from the inlet end 152 flowing through the inner volume 154 towards the outlet end 158. The second flow 104 enters the static mixer 150 at an inlet 108 of the second flow conduit 155 to the annular chamber 159 and then enters the inner volume 107 of the mixing conduit 100 to mix with the first flow 105 through the plurality of jet openings 102. A mixed flow 157 exits the static mixer 150 through the outlet end 158 of the first-flow conduit 153.

The mixing conduit 100 isolates the first-flow conduits 153 from the second-flow conduit 155 so that the second flow 104 can only mix with the first flow 105 via the plurality of jet openings 102 in the mixing conduit 100. The parameters and structures of the mixing conduit 100 may be designed to obtain a desirable mixing result.

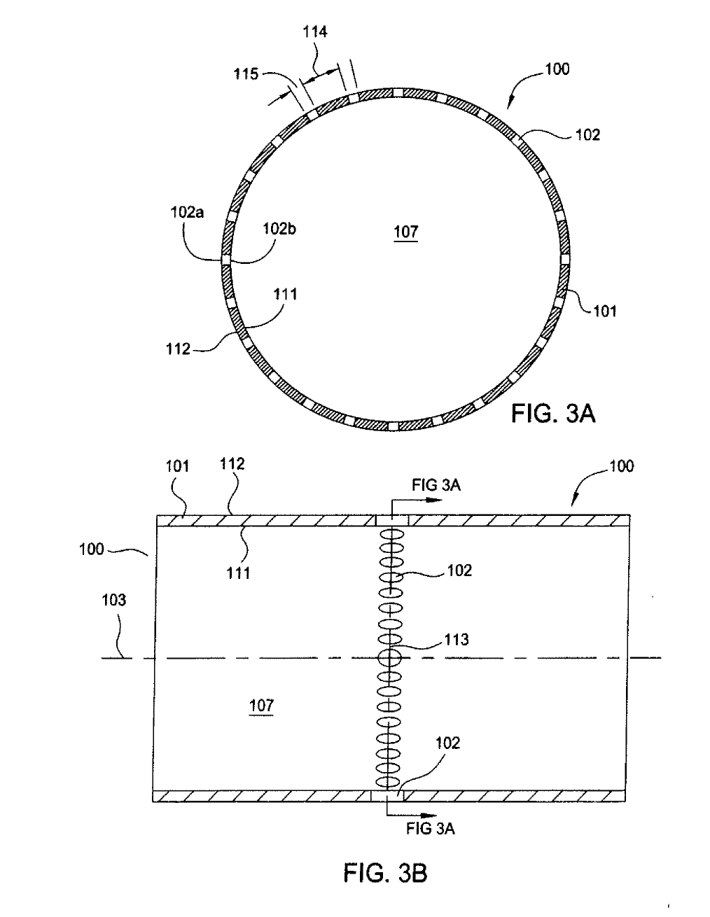

FIG. 3A is a sectional view of the mixing conduit 100 according to one embodiment of the present invention. FIG. 3B is a side view of the mixing conduit 100. The sidewall 101 of the mixing conduit 100 surrounds the central axis 103 and forms the inner volume 107. The sidewall 101 is rotationally-symmetric about the central axis 103 to maximize the strength of the sidewall 101 without increasing the thickness of the sidewall 101 to withstand radial stress and axial stress loaded on the sidewall 101 during operation, such as stresses to the sidewall 101 caused by the differential pressure between the first flow 105 and the second flow 104 in the static mixer 150 of FIG. 2. In one embodiment, the sidewall 101 has a substantially cylindrical cross section and the inner volume 107 is a cylindrical volume. In one embodiment, at least one of an inner surface 111 and an outer surface 112 has a circular cross section.

The plurality of jet openings 102 are formed through the sidewall 101. In one embodiment, the plurality of jet openings 102 are evenly distributed along the sidewall 101 with in a plane 113 substantially perpendicular to the central axis 103. Each jet opening 102 may be circular, elliptical, polyngonal, or other suitable shape. Each jet opening 102 has a first end 102a on the outer surface 112 and a second end 102b on the inner surface 111. In one embodiment, the first end 102a and the second end 102b of each jet opening 102 have the same shape rendering a cylindrical opening. In an alternative embodiment, each jet opening 102 may be tapered having the first end 102a wider than the second end 102b.

The number of jet openings 102 is set to obtain improved mixing results and reduce undesired by-product. The number of jet openings 102 may be affected by various parameters, such as the diameter of the mixing conduit 100, the average velocity of the first flow 105 entering the mixing conduit 100, and the ratio of flow rates of the first flow 105 and the second flow 104. When the mixing conduit 100 is used in phosgenation processes, the number of jet openings 102 for amine flows may be affected by factors such as the diameter of the pipe for incoming phosgene, average velocity of phosgene, and ratio of amine/phosgene flow rates.

According to one embodiment of the present invention, under the same flow rates, performance of the static mixer, such as static mixer 150, may be improved by increasing the number of jet openings 102. The pressure drop through the jet openings 102 is strongly correlated to the total cross-sectional area of these openings. Differential pressure may be maintained by decreasing the jet opening size when increasing the number of jet openings 102. The performance of the static mixer 150 is measured by the yield loss (or percentage of undesired by-products). Not wishing to be bound by theory, smaller streams (obtained by more jet openings) of the second stream mix faster with the first stream than larger streams of the second stream.

However, mechanical integrity and fouling issues may limit the jet openings to be within a reasonable number. For example, the number of jet openings 102 may be limited by geometry of the sidewall 101. In one embodiment, the first end 102a of each jet opening 102 has a width 115, and neighboring jet openings 102 are at a distance 114 away from one another. The ratio of the distance 114 and the width 115 decreases as the number of jet openings 102 increases. The ratio of the distance 114 and the width 115 needs to be maintained above a certain value to ensure that the mixing conduit 100 is structurally sound. This ratio is determined based on the strength of the material used to construct the conduit 100.

Alternatively, the number of jet openings 102 may be determined according a cross section area of the inner volume 107 and total cross sectional area of the plurality of the jet openings 102. The number of plurality of jet openings 102 is maximized to maintain a ratio of the total cross sectional areas of the plurality of jet openings 102 and the cross sectional area of the inner volume 107.

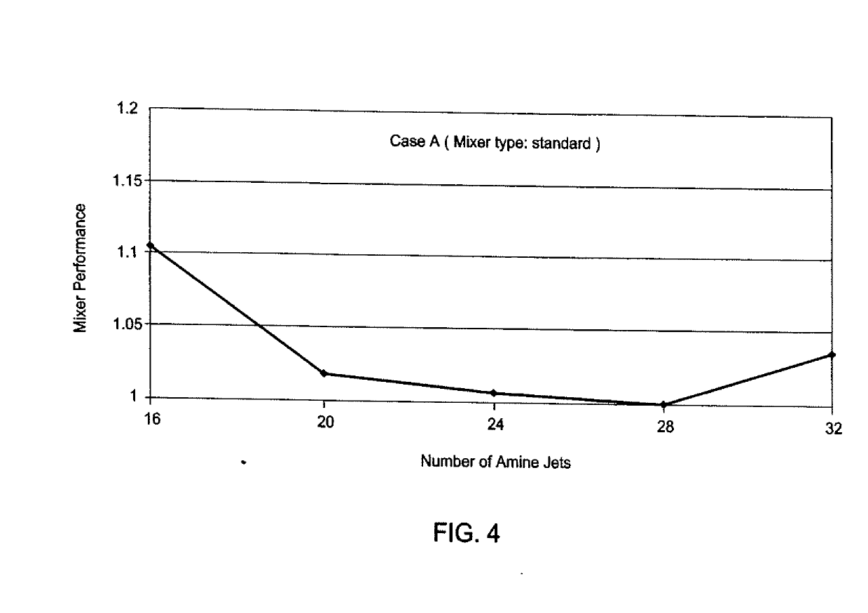

According to embodiments of the present invention, the number of jet openings 102 is between about 22. In another embodiment, the number of jet openings 102 is at least 24. In one embodiment, the number of jet openings 102 is between about 24 to about 32. In another embodiment, the number of jet openings is about 28. FIG. 4 is a graph showing simulated mixer performance of static mixtures similar to the static mixer 150 shown in FIG. 2. This simulation is based on a computer model that has been validated through comparison with experimental data. The simulated process is a phosgene and amine mixing process, where a flow of phosgene enters the first flow conduit 153 and a flow of amine enters the second flow conduit 155 and mixes with the flow of phosgene through the plurality of jet openings 102. In the simulation process, performance of static mixers 150 with a mixing conduit 100 having various number of jet openings 102 formed on a cylindrical sidewall are evaluated. All the mixing process parameters, flow rates of phosgene and amine, and total cross sectional area of the plurality of jet openings 102, the temperatures of process streams are maintained constant while the number of jet openings 102 changes.

The x-axis indicates the number of jet openings. The y-axis indicates the mixer's simulated performance in normalized rate of undesirable by-product. The lower value in y-axis indicates a better performance.

As illustrated in FIG. 4, as the number of jet openings 102 increases from 16 to 28, the simulated performance of the static mixer improves. The performance of the static mixer decreases as the number of jet openings 102 increases to 32. The simulation result in FIG. 4 indicates that for a cylindrical mixing conduit, improved mixing performance may be obtained by increasing the number of jet openings to about 24 to 28. This result is not suggested by the prior art which teaches the number of jet openings within the range of 2 and 20 for a cylindrical mixing conduit.

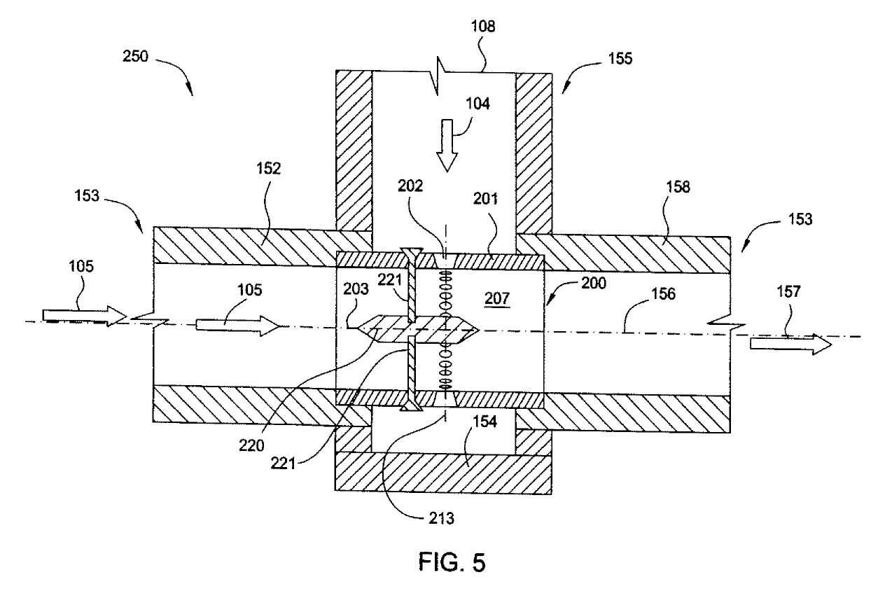

FIG. 5 is a sectional view of a static mixer 250 according to one embodiment of the present invention. The static mixer 250 is similar to the static mixer 150 of FIG. 2 except the static mixer 250 having a mixing conduit 200 in place of the mixing conduit 100.

The mixing conduit 200 comprises a substantially cylindrical sidewall 201 surrounding a central axis 203 and defining an inner volume 207. A plurality of jet openings 202 are formed through the sidewall 201 within a plane 213 substantially perpendicular to the central axis 203. The mixing conduit 200 further comprise a streamlined axial flow obstruction 220 secured to a plurality of spokes 221. The axial flow obstruction 220 may have a cylindrical middle section and tapered ends. In one embodiment, the axial flow obstruction 220 is coaxial with the sidewall 201 and intersects with the plane 213.

The axial flow obstruction 220 reduces cross-sectional area in the mixing conduits 200, thus increasing the velocity of the first flow 105 near the plane 213 where the second flow enters. The axial flow obstruction 220 eliminates the first flow 105 near the central axis 203 to improve mixing in the situations when the flow from jet openings 202 cannot reach the central axis 206. Additionally, the axial flow obstruction 220 also provides obstruction along a longitude of the mixing conduit 200 so that the effect of obstructions from the spokes 221 and axial flow obstruction 220 extends further downstream. Detailed description of the axial flow obstruction design may be found in U.S. patent application Ser. No. 12/725,262 filed on Mar. 16, 2010, by at least a partial common inventorship, which is incorporated herein by reference.

According to one embodiment of the present invention, the number of jet openings 202 is between about 22 to about 50. In another embodiment, the number of jet openings 202 is at least 24. In one embodiment, the number of jet openings 202 is between about 24 to about 36. In another embodiment, the number of jet openings 202 is about 28.

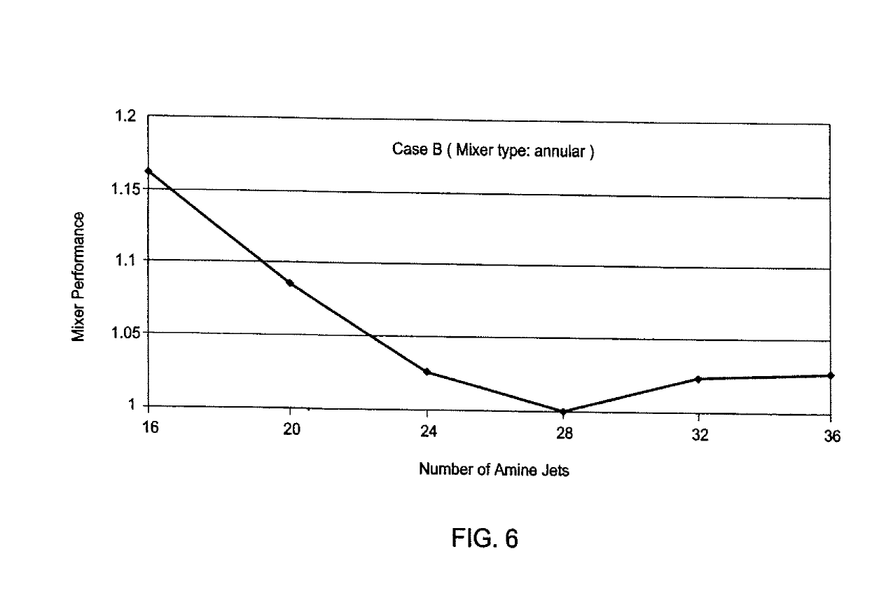

FIG. 6 is a graph showing simulation performance of static mixers 250 shown in FIG. 5. The simulation is made using models that have been validated through comparison with experimental data. The simulated process is a phosgene and amine mixing process, where a flow of phosgene enters the first flow conduit 153 and a flow of amine enters the second flow conduit 155 and mixes with the flow of phosgene through the plurality of jet openings 202. In the simulated process, performance of static mixers 250 with a mixing conduit 200 having various number of jet openings 202 formed on a cylindrical sidewall are evaluated. The flow rates of phosgene and amine, and total cross sectional area of the plurality of jet openings 202 are maintained constant while the number of jet openings 202 changes.

The x-axis indicates the number of jet openings. The y-axis indicates the mixer's performance in normalized rate of undesirable by-product. The lower value in y-axis indicates a better performance.

As illustrated in FIG. 6, as the number of jet openings 502 increases from 16 to 28, the performance of the static mixer improves. The performance of the static mixer gradually decreases as the number of jet openings 202 increases to 36. The simulation result in FIG. 6 indicates that for a cylindrical mixing conduit with an axial flow obstruction, optimal mixing performance may be obtained by increasing the number of jet openings to about 24 to 36.

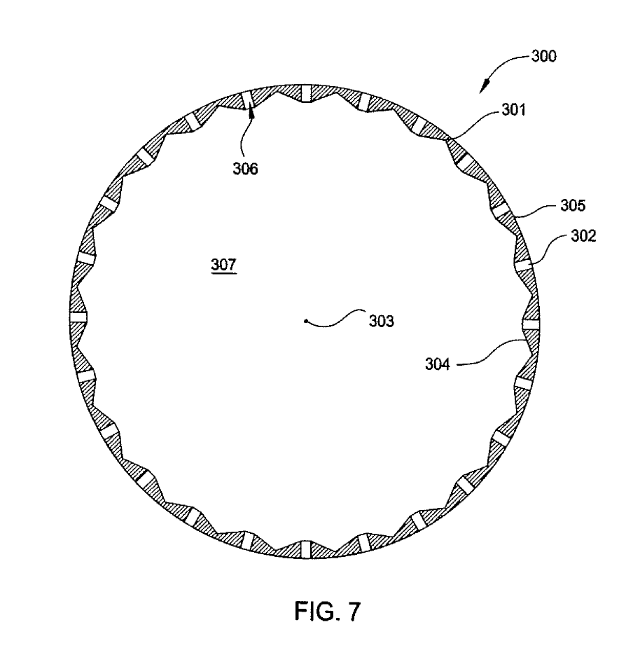

FIG. 7 is a sectional view of a mixing conduit 300 according to another embodiment of the present invention. The mixing conduit 300 may be used in place of the mixing conduit 100 or 200 in a static mixer 150 or 250.

The mixing conduit 300 has a sidewall 301 defining an inner volume 307. The sidewall 301 is rotational symmetric about a central axis 303. The inner volume 307 extends along the central axis 303. A plurality of jet openings 302 are formed through the sidewall 301. The plurality of jet openings 302 may be evenly distributed along the circumference of the sidewall 301. In one embodiment, the number of jet openings 302 may be above about 22. In another embodiment, the number of jet openings 302 is at least 24. In another embodiment, the number of jet openings 302 is between about 24 to about 32.

The sidewall 301 has an outer surface 305 and an inner surface 304. The outer surface 305 is a cylindrical surface having a circular cross section. The inner surface 304 may have a non-circular cross section forming grooves 306 along the direction of central axis 303 for directing the flow therein. The cylindrical outer surface 305 provides advantages of a cylindrical sidewall and the polygonal inner surface 304 provides effects towards the flow. The inner surface 304 may have other shapes to obtain desired effects on the flow.

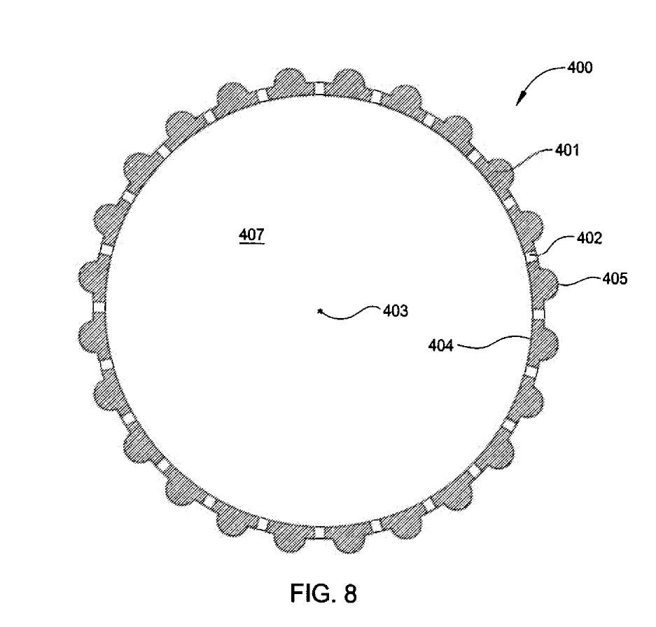

FIG. 8 is a sectional view of a mixing conduit 400 according to another embodiment of the present invention. The mixing conduit 400 may be used in place of the mixing conduit 100 or 200 in a static mixer 150 or 250.

The mixing conduit 400 has a sidewall 401 defining an inner volume 407. The sidewall 401 is rotational symmetric about a central axis 403. The inner volume 407 extends along the central axis 403. A plurality of jet openings 402 are formed through the sidewall 401. The plurality of jet openings 402 may be evenly distributed along the circumference of the sidewall 401. In one embodiment, the number of jet openings 402 may be above 22. In another embodiment, the number of jet openings 402 is at least 24. In another embodiment, the number of jet openings 402 is between about 24 to about 32.

The sidewall 401 has an outer surface 405 and an inner surface 404. The outer surface 405 has a non-circular cross section. The inner surface 404 is a cylindrical surface having a circular cross section. The cylindrical inner surface 404 provides advantages of a cylindrical sidewall.

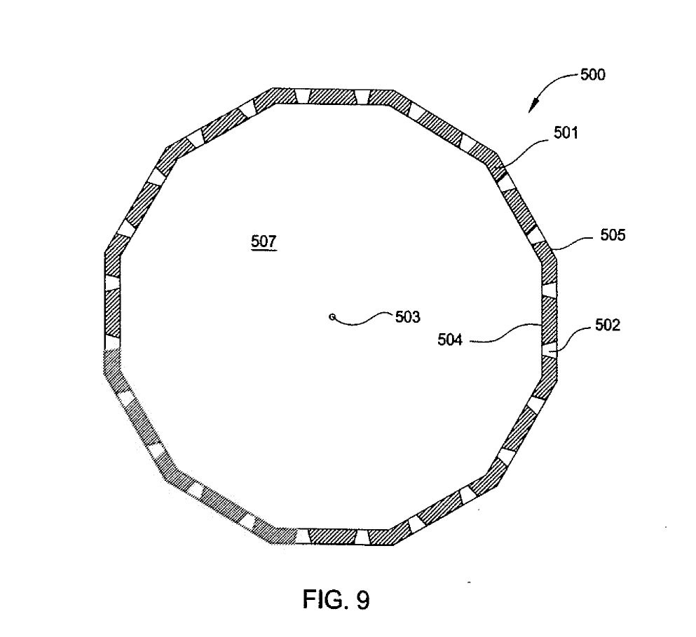

FIG. 9 is a sectional view of a mixing conduit 500 according to another embodiment of the present invention. The mixing conduit 500 may be used in place of the mixing conduit 100 or 200 in a static mixer 150 or 250.

The mixing conduit 500 has a sidewall 501 defining an inner volume 507. The sidewall 501 is symmetric about a central axis 503. The inner volume 507 extends along the central axis 503. A plurality of jet openings 502 are formed through the sidewall 501. The plurality of jet openings 502 may be evenly distributed along the circumference of the sidewall 501. In one embodiment, each jet opening 502 may be tapered. In one embodiment, the number of jet openings 502 may be above 32. In another embodiment, the number of jet openings 502 is at least 24. In another embodiment, the number of jet openings 502 is between about 24 to about 32.

The sidewall 501 has an outer surface 505 and an inner surface 504. Both the outer surface 505 and the inner surface 504 have a non circular cross section. For example, both the outer surface 505 and the inner surface 504 have a cross section of a regular polygon. The regular polygonal sidewall 501 may provide structural advantages similar to a cylindrical sidewall.

Embodiments of the present invention provide static mixers having a substantially cylindrical mixing conduit with increased number of jet openings, which improves mixing performance. The design on increased number of jet openings allows fewer simultaneously operating mixers in a system, thus reducing the overall operating cost.

Embodiment of the present invention may be used as an a direct extension of other designs in a static mixer, such as a mixers with tapered jet opening, complex jet openings, phosgene stream diverters. For example, the jet openings may be combined with tapered apertures described in U.S. patent application Ser. No. 11/658,193, filed Jul. 7, 2005, published as US Publication 2008/0087348 and granted as U.S. Pat. No. 7,901,128, or jet openings described in U.S. patent application Ser. No. 12/725,266 filed on Mar. 16, 2010, or flow obstructions described in U.S. Provisional Application No. 61/387,229 filed on Sep. 28, 2010, which have a least partial common inventorship and are incorporated herein by reference.

While the foregoing is directed to embodiments of the invention, other and further embodiments of the invention may be devised without departing from the basic scope thereof.

What is claimed is:

1. A mixing conduit, comprising:
a sidewall surrounding an axis and enclosing an inner volume along the axis, wherein the inner volume has two openings on opposite ends allowing an axial stream to enter and exit the inner volume along the axis, the sidewall has an inner surface facing the inner volume and an outer surface facing an exterior volume, at least one of a cross section of the inner surface and a cross section of the outer surface is circular, a plurality of jet openings are formed through the sidewall in a plane perpendicular to the axis, the plurality of jet openings allow lateral streams to flow from the exterior volume into the inner volume, and the number of the plurality of jet openings is greater than 22.

2. The mixing conduit of claim 1, wherein the number of jet openings is between about 22 and about 50.

3. The mixing conduit of claim 1, wherein the number of the plurality of jet openings is greater than 24.

4. The mixing conduit of any one of claim 1 wherein the cross section of the inner surface is circular and the cross section of the outer surface is non-circular.

5. The mixing conduit of any one of claim 1, wherein the cross section of the outer surface is circular and the cross section of the inner surface is non-circular.

6. The mixing conduit of any one of claim 1, wherein the cross section of the inner surface is circular and the cross section of the outer surface is circular.

7. The mixing conduit of claim 1, further comprising an axial flow obstruction disposed in the inner volume along the axis, wherein the axial flow obstruction passes the plane in which the plurality of jet openings are formed.

8. The mixing conduit of claim 7, further comprises two or more spokes securing the axial flow obstruction to the sidewall.

9. The mixing conduit of claim 1, wherein each of the plurality of jet openings is tapered having a large opening at the outer surface of the sidewall and a small opening at the inner surface of the sidewall.

10. A mixing conduit, comprising:
a cylindrical sidewall defining a cylindrical inner volume, wherein the inner volume has a first cross sectional area, a plurality of jet openings are formed through the cylindrical sidewall in a plane perpendicular to an axis of the cylindrical inner volume, and the number of plurality of jet openings is maximized to maintain a ratio of a total cross sectional areas of the plurality of jet openings and the first cross sectional area and the physical integrity of the cylindrical wall.

11. The mixing conduit of claim 10, wherein the number of jet openings is between about 22 and about 50.

12. The mixing conduit of claim 11, wherein the number of the plurality of jet openings is greater than 24.

13. A static mixer comprising:
a first flow receiving conduit;
a second flow receiving conduit, wherein the first and second receiving conduits define one or more outer walls of an annular chamber; and
a mixing conduit of claim 1 disposed in a first conduit and forming at least an inner wall of the annular chamber, wherein the annular chamber is in fluid communication with the one or more jet openings of the mixing conduit.

14. A method for mixing comprising:
flowing a first flow along a longitude of the mixing conduit of claim 1; and
flowing a second flow through the one or more jets of the mixing conduit of claim 1.

15. The method of claim 14, wherein the first flow comprises phosgene and the second flow comprises amines.

16. The method of claim 15, wherein the second flow comprises at least one of methylene diphenyl diamine, toluene diamine, and hexamethylene diamine.

17. The method of claim 16, wherein the one or more flow obstructions disposed in the inner volume modifies velocity profiles in the first flow such that an amount of ureas, carbodiimides, and uretonimines formed are less than in a method where no obstructions are disposed in the inner volume.
